(19) United States
(12) Patent Application Publication
(10) Pub. No.: US 2008/0262326 A1
(43) Pub. Date: Oct. 23, 2008

(54) Signal Processing Method and Apparatus for Processing a Physiologic Signal such as a Photoplethysmography Signal

(75) Inventors: Bernard F. Hete, Kittanning, PA (US); Eric J. Ayers, Aliquippa, PA (US)

Correspondence Address: BLYNN L. SHIDELER;THE BLK LAW GROUP, 3500 BROKKTREE ROAD, SUITE 200, WEXFORD, PA, 15090, US

(73) Assignee: STARR LIFE SCIENCES CORP., Oakmont, PA (US)

(21) Appl. No.: 12/106,781

(22) Filed: Apr. 21, 2008

Related U.S. Application Data
(60) Provisional application No. 60/912,923, filed on Apr. 19, 2007.
(60) Provisional application No. 60/938,091, filed on May 15, 2007.

(51) Int. Cl. A61B 5/1455

(52) U.S. Cl. 600/323

(57) ABSTRACT

A signal processing method of processing a physiologic signal, such as a Photoplethysmography Signal having at least some cardiac components and/or respirator components in the physiologic signal, the processing including the steps of: Identifying a potential cardiac and or respiratory components of a physiologic signal wherein the potential cardiac and or respiratory components have a series of peaks and valleys; Calculating a comparison of the durations of a peak to valley sub-component and a valley to peak sub component of the potential cardiac and or respiratory components; and Utilizing the calculated comparison to evaluate the potential cardiac and or respiratory components.

### RELATED APPLICATIONS

The present application claims the benefit of U.S. provisional patent application Ser. No. 60/912,923 entitled “Breath Signal Identification on a Photoplethysmography Signal Using I:E Ratio” filed Apr. 19, 2007.

The present application claims the benefit of U.S. provisional patent application Ser. No. 60/938,091 entitled “Breath Signal Identification on a Photoplethysmography Signal Using I:E Ratio” filed May 15, 2007.

### BACKGROUND OF THE INVENTION

1. Field of the Invention

The present invention relates to signal processing techniques for processing physiologic signals having cardiac components, and more particularly to medical devices and techniques for deriving cardiac and breathing parameters of a subject from extra-thoracic blood flow measurements and for differentiating cardiac and breathing waveforms on the photoplethysmography signal, sometimes references as a photopleth signal, in which the cardiac and breathing waveforms are super-imposed on each other.

2. Background Information

As background, one type of non-invasive physiologic sensor is a pulse monitor, also called a photoplethysmograph, which typically incorporates an incandescent lamp or light emitting diode (LED) to trans-illuminate an area of the subject, e.g. an appendage, which contains a sufficient amount of blood. In the photoplethysmographic phenomenon the light from the light source disperses throughout the appendage and a light detector, such as a photodiode, is placed on the opposite side of the appendage to record the received light for transmisive type devices or on the same side of the appendage for reflective type devices. Due to the absorption of light by the appendage's tissues and blood the intensity of light received by the photodiode is less than the intensity of light transmitted by the LED. Of the light that is received, only a small portion (that effected by pulsatile arterial blood), usually only about two percent of the light received, behaves in a pulsatile fashion. The beating heart of the subject, and the breathing of the subject as discussed below, creates part of this pulsatile behavior. The “pulsatile portion light” is the signal of interest and effectively forms the photoplethysmograph. The absorption described above can be conceptualized as AC and DC components. The arterial vessels change in size with the beating of the heart and the breathing of the patient. The change in arterial vessel size causes the path length of light to change from dₘᵢₙ to dₘₐₓ. This change in path length produces the AC signal on the photo-detector, I_{L} to I_{H}. The AC Signal is, therefore, also known as the photo-plethysmograph.

The absorption of certain wavelengths of light is also related to oxygen saturation levels of the hemoglobin in the blood transfusing the illuminated tissue. In a similar manner to the pulse monitoring, the variation in the light absorption caused by the change in oxygen saturation of the blood allows for the sensors to provide a direct measurement of arterial oxygen saturation, and when used in this context the devices are known as oximeters. The use of such sensors for both pulse monitoring and oxygenation monitoring is known and in such typical uses the devices are often referred to as pulse oximeters.

These devices are well known for use in humans and large mammals and are described in U.S. Pat. Nos. 4,621,643; 4,700,708 and 4,830,014 which are incorporated herein by reference. See also U.S. the following United States Published Patent Applications which are incorporated herein by reference:

PUB. APP.
Title

NO.
1 20080072906 PULSE OXIMETER BASED TECHNIQUES FOR CONTROLLING ANESTHESIA LEVELS AND VENTILATION LEVELS IN SUBJECTS
2 20080064936 LOW POWER PULSE OXIMETER
3 20080058621 Methods and Devices for Countering Grativity Induced Loss of Consciousness and Novel Pulse Oximeter Probes
4 20080045822 Optical Fibre Catheter Pulse Oximeter
5 20080039701 Dual-mode pulse oximeter
6 20080030468 Systems and methods for acquiring calibration data usable in a pulse oximeter
7 20080009691 REUSABLE PULSE OXIMETER PROBE AND DISPOSABLE BANDAGE APPARATII
8 20070244377 PULSE OXIMETER SLEEVE
9 20070208242 Selection of ensemble averaging weights for a pulse oximeter based on signal quality metrics
10 20070156039 Pulse oximeter and sensor optimized for low saturation
11 20070100219 Single use pulse oximeter
12 20070100218 Single use pulse oximeter
13 20070073119 Wireless network connected pulse oximeter
14 20070049812 Time-segmented pulse oximetry and pulse oximeter performing the same
15 20070027380 Shunt barrier in pulse oximeter sensor
16 20070027379 Shunt barrier in pulse oximeter sensor
17 20070027378 Shunt barrier in pulse oximeter sensor
18 20070027377 Shunt barrier in pulse oximeter sensor
19 20070027376 Probe adapted to be used with pulse oximeter
20 20070021663 Shunt barrier in pulse oximeter sensor
21 20070021662 Shunt barrier in pulse oximeter sensor
22 20070021661 Shunt barrier in pulse oximeter sensor
23 20070021660 Shunt barrier in pulse oximeter sensor
24 20070021659 Shunt barrier in pulse oximeter sensor
25 20070015982 Shunt barrier in pulse oximeter sensor
26 20060258926 Systems and methods for acquiring calibration data usable in a pulse oximeter
27 20060247507 LIGHT TRANSMISSION SIMULATOR FOR PULSE OXIMETER
28 20060211929 Pulse oximeter and sensor optimized for low saturation
29 20060195280 Pulse oximeter with separate ensemble averaging for oxygen saturation and heart rate
30 20060195027 Pulse oximeter and sensor optimized for low saturation
31 20060195026 Pulse oximeter and sensor optimized for low saturation
32 20060189862 Pulse oximeter and sensor optimized for low saturation
33 20060183988 Pulse oximeter with parallel saturation calculation modules
34 20060173257 Sleep evaluation method, sleep evaluation system, operation program for sleep evaluation system, pulse oximeter, and sleep support system
35 20060030763 Pulse oximeter sensor with piece-wise function
36 20050197793 Pulse oximeter with separate ensemble averaging for oxygen saturation and heart rate
37 20050197552 Pulse oximeter with alternate heart-rate determination
38 20050197551 Stereo pulse oximeter
39 20050197549 Selection of ensemble averaging weights for a pulse oximeter based on signal quality metrics
40 20050187450 LED forward voltage estimation in pulse oximeter
41 20050124871 Pulse oximeter with parallel saturation calculation modules
42 20050113655 Wireless pulse oximeter configured for web serving, remote patient monitoring and method of operation
43 20050101848 Pulse oximeter access apparatus and method
44 20050065417 Dual-mode pulse oximeter
45 20050065414 Pulse oximeter system
46 20050049469 Pulse oximeter
47 20050020894 Oversampling pulse oximeter
48 20040204639 Pulse oximeter and sensor optimized for low saturation
49 20040181134 Pulse oximeter with parallel saturation calculation modules
50 20040181133 Low power pulse oximeter
51 20040171920 Pulse oximeter sensor with piece-wise function
52 20040158135 Pulse oximeter sensor off detector
53 20040158134 Pulse oximeter probe-off detector
54 20040122301 Parameter compensated pulse oximeter
55 20040059209 Stereo pulse oximeter
56 20040054269 Pulse oximeter
57 20040034294 Pulse oximeter
58 20040034293 Pulse oximeter with motion detection
59 20030163033 Apparatus and method for monitoring respiration with a pulse oximeter
60 20030144584 Pulse oximeter and method of operation
61 20030139656 Pulse oximeter probe-off detection system
62 20030069486 Low power pulse oximeter
63 20030028357 Reduced cross talk pulse oximeter
64 20030028085 Low power pulse oximeter
65 20030009092 Reusable pulse oximeter probe and disposable bandage apparatus
66 20020198442 Pulse oximeter
67 20020177762 Oversampling pulse oximeter
68 20020173708 Shunt barrier in pulse oximeter sensor
69 20020161291 Pulse oximeter user interface
70 20020137995 Detection of sensor off conditions in a pulse oximeter
71 20020082489 Pulse oximeter and sensor optimized for low saturation
72 20020082488 Stereo pulse oximeter
73 20020072660 Pulse oximeter probe-off detector
74 20020042558 Pulse oximeter and method of operation
75 20020038082 Pulse oximeter sensor with widened metal strip
76 20020035318 Pulse oximeter sensor with piece-wise function
77 20010029325 Reusable pulse oximeter probe and disposable bandage method
78 20010029324 Pacifier pulse oximeter sensor
79 20010000790 Shunt barrier in pulse oximeter sensor

Current commercial pulse oximeters do not have the capability to measure breath rate or other breathing related parameters other than blood oxygenation. An indirect (i.e. not positioned within the airway or airstream of the subject), non-invasive method for measuring breath rate is with impedance belts.

It is an object of the present invention to minimize the drawbacks of the existing systems and to provide medical devices and techniques for deriving cardiac and breathing parameters of a subject from extra-thoracic blood flow measurements and for differentiating cardiac and breathing waveforms on the photopleth signal in which they are super-imposed on each other.

### SUMMARY OF THE INVENTION

It is noted that, as used in this specification and the appended claims, the singular forms “a,” “an,” and “the” include plural referents unless expressly and unequivocally limited to one referent. For the purposes of this specification, unless otherwise indicated, all numbers expressing any parameters used in the specification and claims are to be understood as being modified in all instances by the term “about.” All numerical ranges herein include all numerical values and ranges of all numerical values within the recited numerical ranges.

The various embodiments and examples of the present invention as presented herein are understood to be illustrative of the present invention and not restrictive thereof and are non-limiting with respect to the scope of the invention.

One non-limiting embodiment of the present invention provides a signal processing method of processing a physiologic signal having at least some cardiac components in the physiologic signal, the processing including the steps of: Identifying a potential cardiac component of a physiologic signal wherein the potential cardiac component has a series of peaks and valleys; Calculating a comparison of the durations of a peak to valley sub-component and a valley to peak sub component of the potential cardiac component; and Utilizing the calculated comparison to evaluate the potential cardiac component.

In one non-limiting aspect of the invention the signal includes at least some respiratory components. In one non-limiting aspect of the invention the signal is a Photoplethysmography Signal. In one non-limiting aspect of the invention the calculated comparison is a ratio of the durations of a peak to valley sub-component and a valley to peak sub component of the potential cardiac component. In one non-limiting aspect of the invention the signal the evaluation of the potential cardiac component includes determining whether the calculated ratio is above or below a preset threshold. In one non-limiting aspect of the invention the signal the evaluation of the potential cardiac component includes flagging the potential cardiac component when the calculated ratio fails to satisfy a preset threshold. In one non-limiting aspect of the invention the signal the calculated comparison includes a calculation of at least a portion of the slopes of the sub-components.

A signal within the meaning of the present application is any time varying quantity, and a physiologic signal is a signal including one or more biometric components or bio-parameter components of a subject from which the signal is obtained. Signal processing is the analysis, interpretation, and manipulation of signals. A physiologic signal within the meaning of this application will be made up of biometric components (or waveforms) and noise. The term noise is a generic phrase herein to effectively reference non-biometric components of the signal. Further, the term noise can be used to encompass all other portions of the signal other than the particular biometric component of interest, whereby this “noise” could include biometric components.

Cardiac components within this application will reference signal components that are indicative of (i.e. a biometric of) the subject's cardiac function. In a similar fashion, respiratory components within this application will reference signal components that are indicative of (i.e. a biometric of) the subject's respiratory function.

The durations of a peak to valley sub-component and a valley to peak sub component of a subject signal is simply a measure of the time that it takes for a signal to move from the identified peak to the identified valley, and vice versa. As will be appreciated, the sum of a peak to valley duration and the adjacent valley to peak duration will yield a peak to peak duration. Similarly the sum of the sum of a valley to peak duration and an adjacent peak to valley duration will yield a valley to valley duration. Therefore a comparison of the durations of a peak to valley sub-component and a valley to peak sub component of the signal, can utilize a peak to peak measurement or valley to valley measurement in place of either a peak to valley sub-component or the valley to peak sub component. All of these variations are effectively equivalent in the end result and are intended to be encompassed in the language that defines a comparison of the durations of a peak to valley sub-component and a valley to peak sub component of the signal.

One non-limiting embodiment of the invention provides a signal processing method of processing a physiologic signal having at least some respiratory and some cardiac components in the physiologic signal, the processing including the steps of: Identifying a potential respiratory component of a physiologic signal wherein the potential respiratory component has a series of peaks and valleys; Calculating a comparison of the durations of a peak to valley sub-component and a valley to peak sub component of the potential respiratory component; and Utilizing the calculated comparison to evaluate the potential respiratory component.

In one non-limiting aspect of the present invention the signal is a Photoplethysmography Signal, and the calculated comparison is a ratio of the durations of a peak to valley sub-component and a valley to peak sub component of the potential respiratory component. In one non-limiting aspect of the present invention the evaluation of the potential respiratory component includes determining whether the calculated ratio is above or below a preset threshold. In one non-limiting aspect of the present invention the evaluation of the potential respiratory component includes flagging the potential respiratory component when the calculated ratio fails to satisfy a preset threshold. In one non-limiting aspect of the present invention the calculated comparison includes a calculation of at least a portion of the slopes of the sub-components.

One non-limiting embodiment of the present invention provides a signal processing method of processing a physiologic Photoplethysmography signal having peaks and valleys in the physiologic signal, the processing including the steps of calculating a comparison of the durations of a peak to valley sub-component and a valley to peak sub component of the physiologic signal, and utilizing the calculated comparison to evaluate the physiologic signal. One non-limiting embodiment of the present invention provides that the physiologic signal is of extra thoracic blood flow, and wherein the physiologic signal is of a small animal such as a mouse.

These and other advantages of the present invention will be clarified in the following description of the preferred embodiments wherein like reference numerals represent like elements throughout.

### BRIEF DESCRIPTION OF THE DRAWINGS

FIG. 1 is a representation of a display screen with a Photoplethysmography physiologic signal displayed thereon with graphical representations of the signal processing according to one aspect of the present invention;

FIG. 2 is a representation of a display screen with another Photoplethysmography physiologic signal displayed thereon with graphical representations of the signal processing according to one aspect of the present invention and of signal flagging in accordance with one aspect of the present invention;

FIG. 3 is a representation of a display screen with another Photoplethysmography physiologic signal displayed thereon; and

FIG. 4 is a representation of a display screen with another Photoplethysmography physiologic signal displayed thereon with signal flagging in accordance with one aspect of the present invention.

### DESCRIPTION OF THE PREFERRED EMBODIMENTS

Pulse oximeters have long been used to provide heart rate measurements as well as blood oxygenation of a subject. A measurement of breath rate from a pulse oximeter was first made commercially available in 2005 by the assignee of the present application, Starr Life Sciences and is provided in the MouseOx™ device that was particularly designed for use with small mammals, namely rats and mice. In this device the breath rate is obtained by screening out the frequency band around the heart rate point on the Fast Fourier Transform (known as FFT) that is used to identify the heart rate. The next largest amplitude to the left (or lower frequency) of the heart rate rejection band on the FFT was considered to be the breath rate. The value is then simply averaged then displayed on the screen to the user.

Although useful there was room to improve this calculation methodology to assure consistent accurate results. One of the difficulties associated with obtaining arterial oxygen saturation using a pulse oximeter is that the breathing waveform can sometimes dominate the photoplethysmography (photopleth) signal, which can cause the software algorithms to incorrectly choose breathing as the cardiac signal. Such a choice results in the oximeter incorrectly displaying breath rate as heart rate. Additionally, since oxygen saturation is calculated based on knowing light transmission at systole and diastole points on the cardiac-derived photopleth signal, a conventional pulse oximeter device can incorrectly calculate oxygen saturation. It is possible to calculate oxygen saturation from the breathing signal, but if the breathing signal is at least partially derived from physical motion of the LED/photodiode sensor pair, the measurement can be incorrect. It is thus required that oxygen saturation be calculated from the cardiac photopleth signal.

The difficulty associated with differentiating cardiac and breathing waveforms on the photopleth signal is that they are super-imposed on each other in the incoming raw signal. Usually, the cardiac signal is much stronger and can be easily discerned, but this may not always be the case. Furthermore, if the signals are inherently very small, as is the case when the sensor is located on a rodent tail, or there is substantial noise on the signal, the ability to differentiate cardiac and breath signals can be very difficult.

After having observed many photopleth signals exemplary of each phenomenon, the applicants note that there is a difference between the general shapes of the breathing and cardiac waveforms. These differences can be explained based on the expected changes in light absorption of the photodiode resulting from the physiological response of the peripheral blood flow at the sensor site to cardiac and respiratory inputs.

In the case of normal cardiac pumping, the contraction or systolic phase of the cardiac cycle is highly dynamic and occurs very quickly, in comparison to the filling or diastolic phase of the cardiac cycle, which lasts longer. This is due to the highly dynamic and active force of contraction to expel blood from the cardiac chambers. The filling, or refractory period is passive, resulting in a longer duration relative to that for ejection.

Breathing cycles behave similarly. The inspiratory phase, which is driven by the active contraction of the diaphragm, occurs much quicker than the expiratory phase, which, under normal sedentary breathing, results from passive recoil of the chest wall. In summary, the contractile phase of the cardiac cycle and the inspiratory phase of the breathing cycle are actively driven and have a shorter duration than the corresponding cardiac filling and expiratory phases, respectively.

In respiratory physiology, the temporal ratio of this phasic differentiation is known as the inspiratory to expiratory ratio or symbolically, I:E. We can use this notation to refer to both the respiratory inspiration to expiration ratio, as well as the contraction (C) to filling (F) ratio. Further, the inspiratory phase of respiration and the contraction phase of cardiac function can be categorized as the active phase of these cycles as noted above. Within the meaning of this application the expiratory phase of respiration and the filling phase of cardiac function are considered the passive phase. To be precise the expiratory phase of respiration can, in certain circumstances, have active components, but for the purpose of this application it is sufficient to categorize this as a passive phase.

Cardiac-Generated Photopleth Signals

Although these two types of cyclic physiological functions have similar temporal characteristics, they differ substantially in their effect on light transmission through tissue. During the systolic portion of the cardiac cycle, blood is pumped from the heart to the periphery. As the blood reaches the sensor location, it causes the local arterial vessels to dilate, which causes an increase in light absorption, and a consequent decrease in light transmission from the LEDs to the photodiode. The result of this vascular dilation is to cause a reduction in signal strength of the photopleth signal during systole.

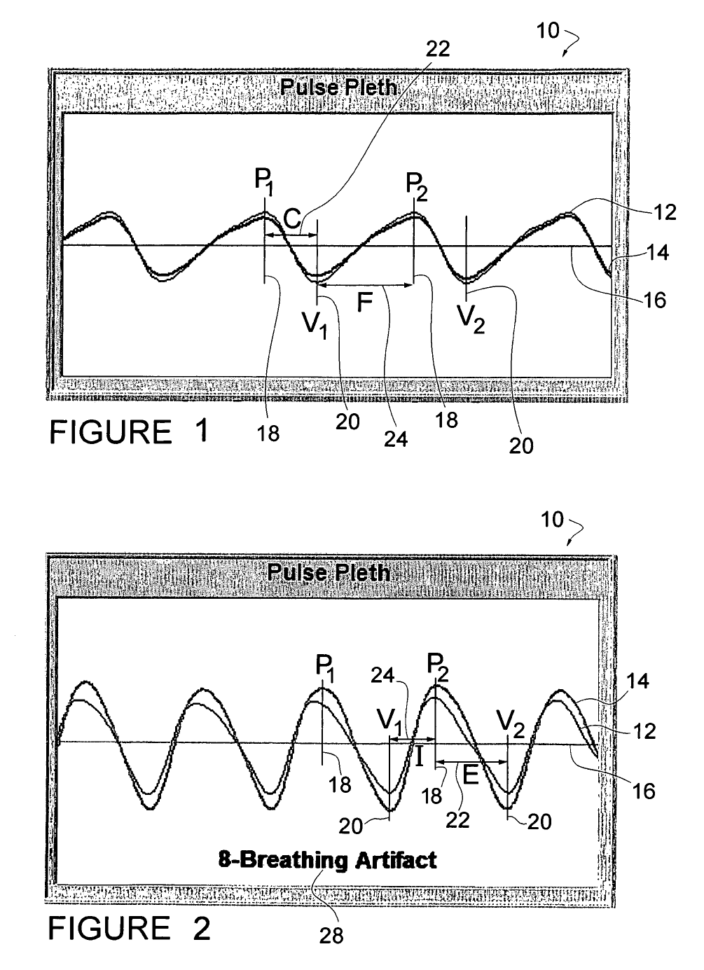

During diastole, the opposite effect occurs. As the blood passes from the arteries, which are not being filled in this phase, through the capillary bed and returns to the heart through the venous system, the local arterial vessels decrease in diameter, which reduces light absorption and increases light transmission. The result is an increase in the signal strength of the photopleth signal during diastole. These phenomena are demonstrated in FIG. 1.

FIG. 1 is a representation of a display screen 10 with a Photoplethysmography physiologic signal displayed thereon in the form of traces 12 and 14, with graphical representations of the signal processing according to one aspect of the present invention. Photopleth signals from red 12 and infrared 14 LEDs received by the photodiode are graphically illustrated on a zero or base axis 16. The oscillations in the traces 12 and 14 of FIG. 1 are typical of those caused by cardiac pulsations. The down stroke occurs during the contraction phase (C), while the temporally longer up stroke occurs during the filling phase (F).

Respiratory-Generated Photopleth Signals

Cyclic respiratory input actually causes the exact opposite effect on received light as that from cardiac input. Breathing inspiratory effort is caused by contraction of the diaphragm, which causes it to be pulled down, away from the lungs, causing a negative pressure in the thorax. This negative pressure gradient draws air into the lungs via vacuum. However, the presence of this negative pressure gradient also acts on the great arteries in the thoracic cavity by exerting external pressure on them. When the intrathoracic pressure is negative, as is the case during inspiration, the great arteries are dilated, which causes blood flow to the periphery to be reduced because blood that would normally have traveled to the periphery must now fill the new intra-arterial volume created in response to the negative pressure gradient in the thoracic cavity. The result is to reduce light absorption and increase the photopleth signal 12, 14 strength during inspiration.

In like manner, during sedentary exhalation, the intra-thoracic pressure is slightly positive, which pushes on the great arteries, causing additional blood to be expelled into the periphery. This effect is greatly exacerbated when breathing becomes labored, and accessory muscles are used to assist in expiration. These phenomena are demonstrated in FIG. 2, which is a representation of a display screen 10 with another Photoplethysmography physiologic signal 12, 14 displayed thereon with graphical representations of the signal processing according to one aspect of the present invention and of signal flagging 28 in accordance with one aspect of the present invention

In FIG. 2 the Photopleth signals from red 12 and infrared 14 LEDs received by the photodiode are shown. The oscillations in the traces 12 and 14 in this figure are typical of those caused by respiratory pulsations. The up stroke occurs during the inspiratory phase, while the temporally longer down stroke occurs during the expiratory phase.

In summary, during inspiration, blood flow to the periphery is reduced, causing increased light transmission to the photodiode, while during expiration, blood flow to the periphery is increased, causing decreased light transmission in trace 12, 14 to the photodiode.

Comparison of Cardiac and Breathing Photopleth Signals

Recall that decreased blood flow to the periphery causes an increase in the photopleth signal strength in trace 12 or 14, while increasing blood flow to the periphery causes a decrease in the strength of the photopleth signal 12 or 14. Recall also that respiratory inspiration and cardiac contraction are similar in that they both occur quicker than their complementary phases. However, as we have just described, the effect of respiratory inspiration and cardiac contraction are opposite with regard to the resulting light transmission. Inspiration causes an increase in light transmission (because of the reduced blood flow to the periphery) while cardiac contraction causes a decrease in light transmission (because of the increased blood flow to the periphery). The complementary phase of each also has the opposite effect on light transmission. Respiratory expiration causes a reduction in light transmission at the periphery (because of the increased blood flow to the periphery), while cardiac filling causes an increase in light transmission at the periphery (because of the decreased arterial blood flow to the periphery).

This reality can be seen by comparing FIGS. 1 and 2. In FIG. 1, in the shorter contraction phase, the photopleth signal 12, 14 decreases, while in FIG. 2, in the shorter inspiratory phase, the photopleth signal 12, 14 increases. The opposite is true for the filling phase of FIG. 1, in which the photopleth signal 12, 14 increases, and for the expiratory phase in FIG. 2, in which the photopleth signal 12, 14 decreases. In the following table, a summary of the differences between cardiac and respiratory input is shown.

| | | | Peripheral | |
|---|---|---|---|---|
| | | | Arterial | |
| | | Relative | Blood | Photopleth |
| Cycle | Phase | Duration | Flow | Signal |
| Cardiac Cycle | Contraction | Short | ↑ | ↓ |
| | Filling | Long | ↓ | ↑ |
| Respiratory | Inspiratory | Short | ↓ | ↑ |
| Cycle | Expiratory | long | ↑ | ↓ |

Implementation of an Algorithm to Identify Breathing Photopleth Signals

Recall that pulse oximetry is normally conducted using a photopleth signal 12, 14 derived from cardiac parameters. If breathing effects become dominant, they may be mistaken for the cardiac signal. Thus, we have developed a method whereby we can use the information given above to allow us to identify breathing signals on the photopleth traces 12, 14.

In order to do this, we use the concept of I:E, except that we use the cardiac signal C:F as the reference, since it is the normal condition. To calculate C:F of the cardiac signal, we can simply identify the peaks 18 and valley 20 of the signal as shown in FIGS. 1 and 2. In this figure, the duration from Peak₁ to Valley₁ is denoted as 22 and illustrates the contraction phase, or the “C” phase here or the active phase. Likewise, the duration from Valley₁ to Peak₂ is denoted as 24, and illustrates the filling phase or the “F” phase here or the passive phase.

We can additionally do the same thing by defining the phases of a breathing-derived photopleth signal 12, 14 as shown in FIG. 2. In this figure, the duration from Valley₁ to Peak₂ is denoted as 24 and here illustrates the “inspiratory” or I phase or the active phase. Likewise, the duration from Peak₂ to Valley₂ is denoted as 22 and here illustrates the expiratory or E phase or the passive phase.

Note in these figures that we have aligned the locations of the duration bands (vertical white lines) with the peaks 18 and valleys 20 of the red signal 12. It must be noted that we could just as easily have aligned them with the infrared 14, or we could have aligned them with both red and infrared signals 12 and 14 simultaneously.

It can be seen by comparing FIGS. 1 and 2 that the duration of the active phase is shorter relative to passive in both graphs, but that the direction of the pulse pleth signals 12 and 14 are effectively inverted. Thus, we can see that the slope of the active phase is negative in a cardiac signal, and it is positive in a respiratory signal. Likewise, the slope of the passive phase is positive in a cardiac signal, and it is negative in a respiratory signal. This difference can be used to identify when breathing is present instead of heart rate.

There are a number of means by which this differentiation can be algorithmically implemented. One could simply identify active and and passive phases for either type of signal 12,14 and use the slope of that phase to determine whether one has a breathing or a cardiac signal 12, 14. This would be done by comparing the slope of the shorter active phase to that of the longer passive phase. If the shorter phase slope is positive, the signal is breathing-derived, while if negative, it is cardiac-derived. This same method could be done using the longer duration phase inversely, or using both simultaneously.

There are also a number of techniques that one can use involving identification of peaks 18 and valleys 20. With such a method, one could calculate the peak to valley time 22, then compare that with valley to peak time 24. For example, we can see from FIG. 1 that we calculate the duration 22 between Peak₁ and Valley₁, and compare that with the duration between 24 Valley₁ and Peak₂. If the former duration 22 is shorter than the latter duration 24, the signal is cardiac-derived. Likewise, if the former duration 22 is longer than the latter duration 24, the signal 12, 14 is respiratory-derived. Additionally, one could calculate the duration 24 between Valley₁ and Peak₂, and compare it with the duration between Peak₂ and Valley₂ 22.

Yet another method is to compare a peak to valley duration 22 or a valley to peak duration 24, and compare it with either a valley to valley duration, or a peak to peak duration (which is effectively the sum of 22 and 24). This comparison could be made against a certain preset threshold, η. For instance, the duration 22 of Peak₁ and Valley₁ could be divided by the duration between Valley₁ and Valley₂. If η were assigned a value of say 0.5, then the algorithm could determine breathing and heart-based signals as follows:

$$\begin{matrix}If \frac{{Valley}_{1} - {Peak}_{2}}{{Valley}_{1} - {Valley}_{2}} > 0.5 , then the signal is cardiac . \\ If \frac{{Valley}_{1} - {Peak}_{2}}{{Valley}_{1} - {Valley}_{2}} \leq 0.5 , then the signal is respiratory .\end{matrix}$$

The value of η is actually somewhat arbitrary, as is the assignment of the equal sign in this example. There are a number of ways to implement the method, but the underlying utility is derived from the difference in characteristic behavior of breathing and cardiac-derived photopleth signals, as illustrated in FIGS. 1 and 2.

Alternate Algorithms to Identify Breathing Photopleth Signals

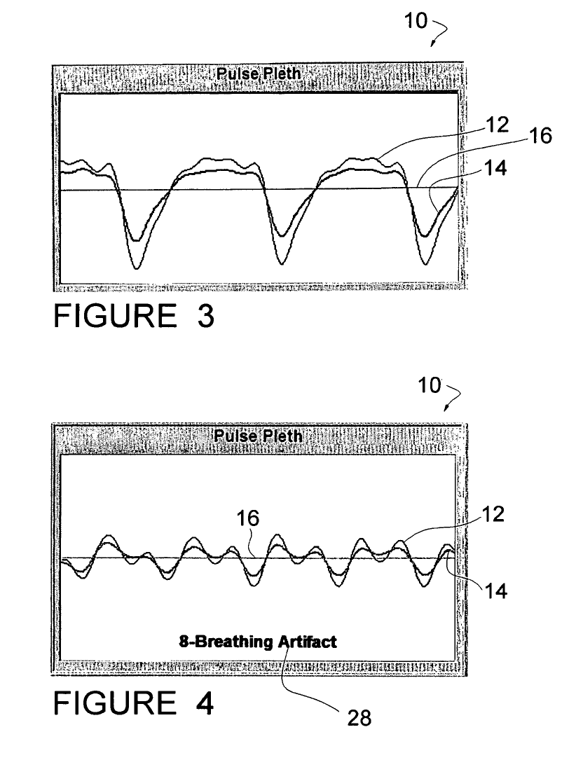

Another method that can be used to differentiate cardiac and breathing signals is through the use of a comparison of the slopes of the up stroke and the down stroke of the photopleth signals. The reason for suggesting this method is that sometimes the cardiac stroke has a long flat portion that may have some ripple on it, as shown in FIG. 3.

In FIG. 3, we are actually looking at a heart rate signal. In such a case, the down stroke should be rapid, while the up stroke is shallower, but because of the long latent period in late diastole, the response flattens out and we have ripple. The peak counting-based algorithms can inadvertently identify one of the peaks from the ripple, and erroneously conclude that we are looking at breath rate rather than heart rate.

To avert this problem, one can find the slopes of the steep part of the curve. In FIG. 3, we see that the slope associated with the signal 12, 14 traversing downward is much steeper than the slope of the portion of the signal 12, 14 that traverses upward. By comparing the relative magnitude of these two slopes, one can assess whether the signal 12, 14 is heart rate or breath rate. In the case of FIG. 3, the steeper slope is on the down stroke, which is associated with systole as described above, and the signal 12, 14 is therefore cardiac.

There are a number of ways to find the region at which the slope can be calculated. This may be tricky because we do not calculate the slope on the flat part of the curve. Thus, we need to find a location that is sufficiently away from the flat portion so that we can get the slope only during the steep portions of the curves.

One method is to take the max and min of the signal 12, 14, then find the midpoint between (generally 16). Wherever the signal 12, 14 crosses the midpoint value 16, the slope can be calculated from points on either side of that midpoint, or on both sides of the midpoint. There are other methods that could involve the crossing of threshold values that are skewed toward either the top or the bottom, or both. The slope could be calculated either between these thresholds, or near one or the other.

Lastly, the slope method described here could be used in conjunction with other methods described above. Multiple methods could be employed using a logical AND or OR.

A further method is to calculate the first moment of area of each section from the peak to the valley and from the valley to the peak. The first moment of area defines a centroid location for the segment and is related to the steepness of the curve. This can provide a robust mathematical approach for implementing the present invention.

A simple approach is merely subtracting the durations 22 and 24 to determine which is longer. It can be seen that there are a number of mathematical relationships to compare the peak to valley and valley to peak durations on the signals 12, 14; including but not limited to addition/subtraction (e.g. (P1toV1)−(V1toP2)), multiplication/division (e.g. (P1toV1)/(V1toV2)), derivative (e.g. slope calculations), integration (moment of area or higher moment of area function), and combinations thereof. Each implementation can have certain advantages, and all of these are within the scope of the present invention.

User-Controlled Differentiation of Experimental Conditions

Another method that can be used to optimize performance of a pulse oximeter in general is to provide a method whereby the user can differentiate their experiment by the use of lack of use of anesthesia, animal species, animal size, etc. Knowledge of this information can allow the designers to optimize measurements for the given conditions. For example, knowledge of the anesthetic state of the animal can allow the digital filtering to be optimized depending on the expectation of motion artifact. There are a large number of applications of such a configuration as it relates to the difficulties associated with measuring oximetry values on small animals.

Implementation of such a method can be done simply by providing one or more buttons on the user interface that would allow the user to choose his conditions. There could also be a default condition if such a choice were not made.

Applications of the Active:Passive Method

The utility of this observation has a number of applications, although the most important is that it allows us to easily differentiate between breathing and cardiac pulse on the photopleth signal. Some of the applications of this utility include the following:

1] An error flag 28 can be thrown when the pulse oximeter algorithms are inadvertently locking on breath rate instead of heart rate in order to make the oxygen saturation measurement. This is demonstrated in FIG. 2 above. The error flag 28 “8-Breathing Artifact” is displayed on the screen 1 0 when the photopleth signal 12, 14 is respiratory-derived. This utility is still present even when both breathing and cardiac input are substantially present on the photopleth signals, as is demonstrated FIG. 4 below.

2] Knowledge of the presence of breathing as the dominant photopleth signal can be used to adjust active filtering in order to enhance the cardiac signal and/or the breathing signal.

3] Knowledge of the I:E/C:F of both breathing and cardiac function can potentially be used as a type of clinical diagnostic marker.

FIG. 4 shows Photopleth signals 12, 14 wherein the large oscillations in the traces are typical of those caused by respiratory pulsations, while the smaller oscillations are typical of those caused by cardiac pulsations. Note that the algorithm still can detect a significant contribution from breathing such that an error flag is thrown. It is also possible to use this technique to adjust active filters to further diminish or eliminate breathing input.

We should finally note that the use of an I:E differentiating method is not limited to transmission pulse oximetry, but could also be used with reflectance pulse oximetry or other sensors obtaining respiratory and cardiac function signals such as respiratory monitors. Although the present invention has been described with particularity herein, the scope of the present invention is not limited to the specific embodiment disclosed. It will be apparent to those of ordinary skill in the art that various modifications may be made to the present invention without departing from the spirit and scope thereof. The scope of the present invention is defined in the appended claims and equivalents thereto.

What is claimed is:

1. A signal processing method of processing a physiologic signal having at least some cardiac components in the physiologic signal, the processing including the steps of:
Identifying a potential cardiac component of a physiologic signal wherein the potential cardiac component has a series of peaks and valleys;
Calculating a comparison of the durations of a peak to valley sub-component and a valley to peak sub component of the potential cardiac component; and
Utilizing the calculated comparison to evaluate the potential cardiac component.

2. The signal processing method according to claim 1 wherein the signal includes at least some respiratory components.

3. The signal processing method according to claim 2 wherein the signal is a Photoplethysmography Signal.

4. The signal processing method according to claim 3 wherein the calculated comparison is a ratio of the durations of a peak to valley sub-component and a valley to peak sub component of the potential cardiac component.

5. The signal processing method according to claim 4 wherein the evaluation of the potential cardiac component includes determining whether the calculated ratio is above or below a preset threshold.

6. The signal processing method according to claim 4 wherein the evaluation of the potential cardiac component includes flagging the potential cardiac component when the calculated ratio fails to satisfy a preset threshold.

7. The signal processing method according to claim 4 wherein the calculated comparison includes a calculation of at least a portion of the slopes of the sub-components.

8. A signal processing method of processing a physiologic signal having at least some respiratory and some cardiac components in the physiologic signal, the processing including the steps of:
Identifying a potential respiratory component of a physiologic signal wherein the potential respiratory component has a series of peaks and valleys;
Calculating a comparison of the durations of a peak to valley sub-component and a valley to peak sub component of the potential respiratory component; and
Utilizing the calculated comparison to evaluate the potential respiratory component.

9. The signal processing method according to claim 8 wherein the signal is a Photoplethysmography Signal.

10. The signal processing method according to claim 9 wherein the calculated comparison is a ratio of the durations of a peak to valley sub-component and a valley to peak sub component of the potential respiratory component.

11. The signal processing method according to claim 10 wherein the evaluation of the potential respiratory component includes determining whether the calculated ratio is above or below a preset threshold.

12. The signal processing method according to claim 10 wherein the evaluation of the potential respiratory component includes flagging the potential respiratory component when the calculated ratio fails to satisfy a preset threshold.

13. The signal processing method according to claim 10 wherein the calculated comparison includes a calculation of at least a portion of the slopes of the sub-components.

14. A signal processing method of processing a physiologic Photoplethysmography signal having peaks and valleys in the physiologic signal, the processing including the steps of Calculating a comparison of the durations of a peak to valley sub-component and a valley to peak sub component of the physiologic signal, and utilizing the calculated comparison to evaluate the physiologic signal.

15. The signal processing method according to claim 14 wherein the calculated comparison is a ratio of the durations of a peak to valley sub-component and a valley to peak sub component of the physiologic signal.

16. The signal processing method according to claim 15 wherein the evaluation of the physiologic signal includes determining whether the calculated ratio is above or below a preset threshold.

17. The signal processing method according to claim 15 wherein the evaluation of the physiologic signal includes flagging the physiologic signal when the calculated ratio fails to satisfy a preset threshold.

18. The signal processing method according to claim 15 wherein the calculated comparison includes a calculation of at least a portion of the slopes of the sub-components.

19. The signal processing method according to claim 15 wherein the physiologic signal is a photophethysmography signal of extra thoracic blood flow of the subject.

20. The signal processing method according to claim 15 wherein the physiologic signal is of a small animal.
